(19) United States
(12) Patent Application Publication
(10) Pub. No.: US 2019/0126058 A1
(43) Pub. Date: May 2, 2019

(54) Method for Inducing In Situ Articular Cartilage Re-Growth Through Chondrocyte Stimulation

(71) Applicant: W Z McCarthy, Salt Lake City, UT (US)

(72) Inventor: W Z McCarthy, Salt Lake City, UT (US)

(21) Appl. No.: 16/182,510

(22) Filed: Nov. 6, 2018

(51) Int. Cl. A61N 5/06; A61M 37/00; A61L 27/54

(52) U.S. Cl. CPC A61N 5/062; A61M 37/00; A61M 37/0092; A61N 2005/067; A61N 2005/0652; A61N 2005/0654; A61L 27/54

(57) ABSTRACT

A method that includes the general steps of injecting a fluid matrix of materials which chondrocytes can use to re-grow cartridge and which encourage chondrocytes to re-grow cartilage, mechanically causing the fluid matrix to travel toward chondrocytes, such as by use of a pressure cuff, an ultrasonic transducer, or other means, and exposing the chondrocytes to photonic stimulation in the range of 165 nm to 280 nm.

### BACKGROUND

Natural aging, joint overuse or abuse, poor nutrition and osteoarthritis leave many adults with worn articular cartilage in their joints with few good options for recovery and a return to normal activity. Particularly active persons, especially runners, gymnasts and construction workers, experience accelerated wear of articular cartridge followed by pain and significant activity restrictions decades before they would otherwise contemplate a sedentary lifestyle.

Worn articular cartridge in adults shows little tendency toward natural re-growth. Most persons experience complete growth of articular cartilage by age 16 and cannot expect substantial growth or re-growth thereafter.

Traditional procedures to address worn articular cartridge include partial or total joint replacement with a prosthetic device. More recently, efforts have been made to grow human cartilage in a lab and then place it in an area of a damaged joint where it is hoped the lab-grown cartilage will attach to the existing joint and allow the patient to recover full use of the damaged joint. In addition, efforts have been made to inject stem cells adjacent articular cartilage in a human joint so that the stem cells will incite a youthful growth phase and allow the joint to recover. None of these prior art options provides a fully satisfactory result for the patient.

### SUMMARY

The invention provides a method for encouraging in situ articular cartilage chondrocyte re-growth. The method does not require use of lab-grown tissue or prosthetic replacement of articular surfaces. The method includes the general steps of:

(a) inject a fluid matrix of materials which chondrocytes can use to re-grow cartridge and which encourage chondrocytes to re-grow cartilage,

(b) mechanically cause the fluid matrix to travel toward chondrocytes, such as by use of a pressure cuff, an ultrasonic transducer, or other means,

(c) expose the chondrocytes to photonic stimulation in the range of 165 nm to 280 nm.

### BRIEF DESCRIPTION OF DRAWINGS

FIG. 1 depicts a human knee joint receiving an injection of a fluid matrix of the invention.

FIG. 2 depicts a human knee with a pressure cuff on it for causing the fluid matrix to reach chondrocytes.

FIG. 3 depicts a human knee with an ultrasonic transducer mounted to it for causing fluid matrix to reach chondrocytes.

FIG. 4 depicts a human knee with a photonic exposure device mounted to it.

### DETAILED DESCRIPTION

Before considering details of the invented treatment method and device, some explanation of the structure and nature of articular cartilage may be helpful.

Articular cartilage is highly specialized connective tissue whose function is to provide a smooth, lubricated surface for articulation and to facilitate the transmission of loads with a low coefficient of friction. Articular cartilage has no blood vessels or nerves and is subject to a harsh biomechanical environment. Consequently, articular cartilage has a limited capacity for intrinsic healing and repair.

Articular cartilage is hyaline cartilage and is generally 2 to 4 mm thick. It consists of a dense extracellular matrix with a sparse distribution of highly specialized cells called chondrocytes. The extracellular matrix is principally composed of water, collagen, and proteoglycans. Articular cartilage is organized into defined zones, namely the superficial zone, the middle zone, the deep zone, and the calcified zone.

Collagen fibers within articular cartilage bear load and provide durability. Chondrocytes produce and maintain the cartilaginous matrix, which consists mainly of collagen and proteoglycans.

Chondrocytes are metabolically active cells that are responsible for development, maintenance, and repair of the collagen fibers of articular cartilage. Chondrocytes originate from mesenchymal stem cells and constitute about 2% of the total volume of articular cartilage. Each chondrocyte establishes a specialized microenvironment and is responsible for the turnover of the extracellular matrix in its immediate vicinity. This microenvironment essentially traps the chondrocyte within its own matrix and so prevents any migration to adjacent areas of cartilage. Rarely do chondrocytes form cell-to-cell contacts for direct signal transduction and communication between cells. They do, however, respond to a variety of stimuli, including growth factors, mechanical loads, piezoelectric forces, and hydrostatic pressures.

Chondrocytes have limited potential for replication, a factor that contributes to the limited intrinsic healing capacity of cartilage in response to injury. Methods are needed to encourage chondrocytes to increase production of collagen for cartilage re-growth. Moving growth resources into proximity with chondrocytes so that they can accelerate collagen production must also be considered.

In order to encourage chondrocytes to produce more collagen, to create a re-growth phase for articular cartridge, materials which encourage and supply the re-growth must be placed in contact with the chondrocytes. The first step is to assemble the materials. The second step is to place them in the general area of the chondrocytes. The third step is to excite or stimulate the chondrocytes so that they use those materials for articular cartilage re-growth.

A chondrocyte supply matrix is created containing materials that chondrocytes can use for re-growth of articular cartilage, and containing materials that tend to encourage that activity.

Materials that chondrocytes can use for re-growth of articular cartridge include lysine, proline, Vitamin C, copper peptides, and various hormones.

Materials that will tend to encourage chondrocytes to re-grow articular cartridge include hyaluronic acid, nanosized carbon tubes, nanoparticulate metals (including Ti, CoCr, Ti6Al4V), and nanoparticulate ceramics (including hydroxyapatite, titanium, aluminum and zinc oxide). These materials, when in contact with chondrocytes, will increase chondrocyte tissue regeneration by promoting the adsorption and bioactivity of proteins (including fibronectin and vitronectin).

Some research has been performed with autologous chondrocyte implantation and mesenchymal stem cell injection, and both can be included in a chondrocyte supply matrix injected into a joint where articular cartridge re-growth is desired. In addition, the matrix can include a naturally-occurring biological tissue growth regulator which encourages cartilage re-growth, such as “Regulator of Cartilage Growth and Differentiation 423,” or RCGD 423.

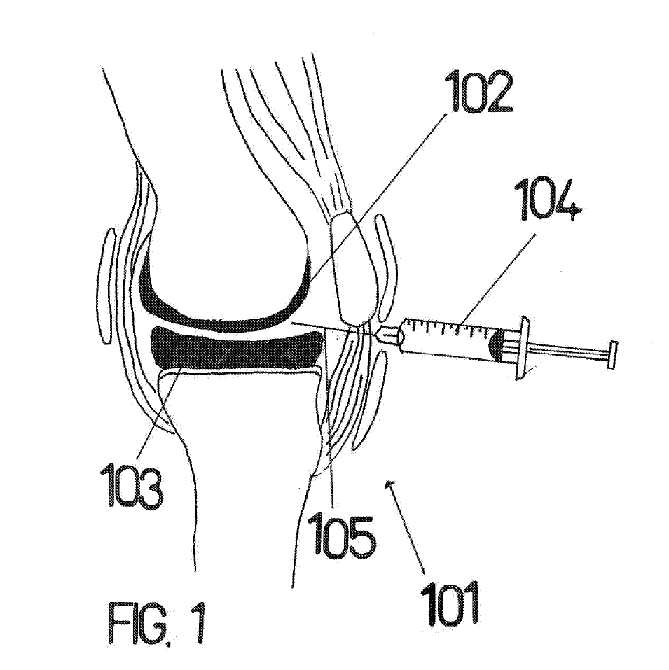

A matrix including some or all of the foregoing can be injected into a joint of a human patient in an area adjacent to articular cartilage. Referring to FIG. 1, a human knee 101 is depicted having articular cartilage 102 and 103. A syringe 104 with needle 105 is used to inject a quantity of fluid matrix into a location adjacent to the articular cartridge 102 and 103 That injection alone, however, is unlikely to encourage much cartilage re-growth due to the avascular nature of articular cartridge. Additional steps must be taken to place materials of the matrix in juxtaposition with chondrocytes, and to encourage the chondrocytes to make use of those materials.

One such will be to do more than wait for the matrix to diffuse itself throughout the synovial fluid of the joint and hope for cartilage re-growth to occur. Some mechanical encouragement is needed to cause the fluid matrix to come into contact with chondrocytes. One option is to utilize a pressurized cuff to apply pressure to the human joint where an injection has occurred, in order to create artificial hydraulic pressure to move the injected matrix into contact with chondrocytes of the articular cartridge. The cuff can use gas or a liquid within a bladder to apply compressive force to the joint in question in order to cause pressurized movement of the injected fluid matrix within the joint.

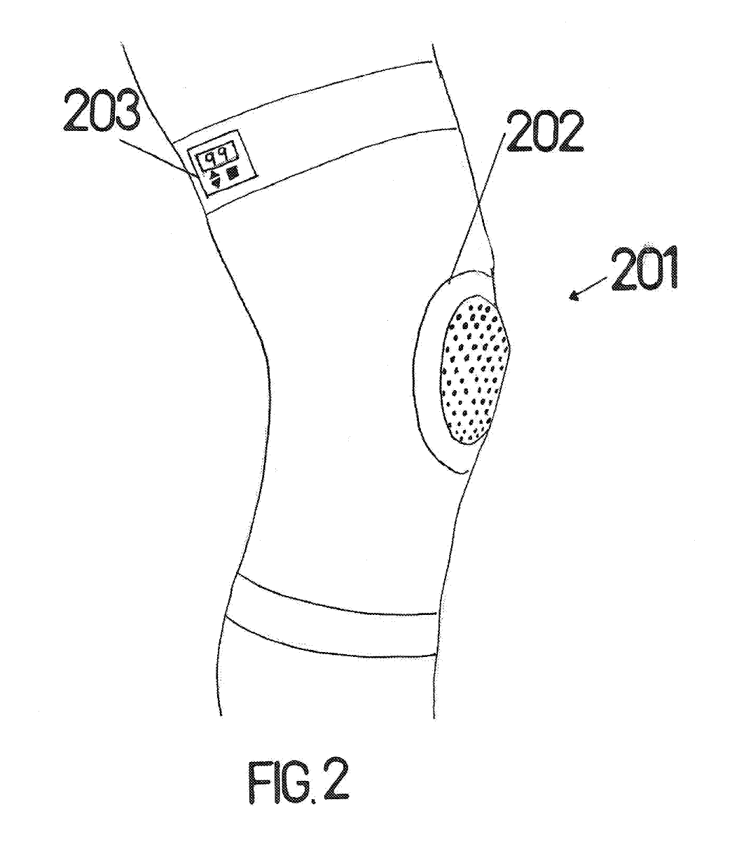

Referring to FIG. 2, a human knee 201 is depicted with a pressure cuff 202 wrapped out it. The cuff 202 can be alternatively pressurized and de-pressurized by a control unit 203 for a desired period of time, and at desired pressures. In such a system it may be beneficial to pressurize the joint to cause the matrix fluid to be pressed against articular cartridge where it can contact chondrocytes and hold it there for a prescribed time period, such as 30 seconds, 1 minute, 5 minutes, etc. Then pressure can be released for another similar time period. The process can be repeated as often as desired.

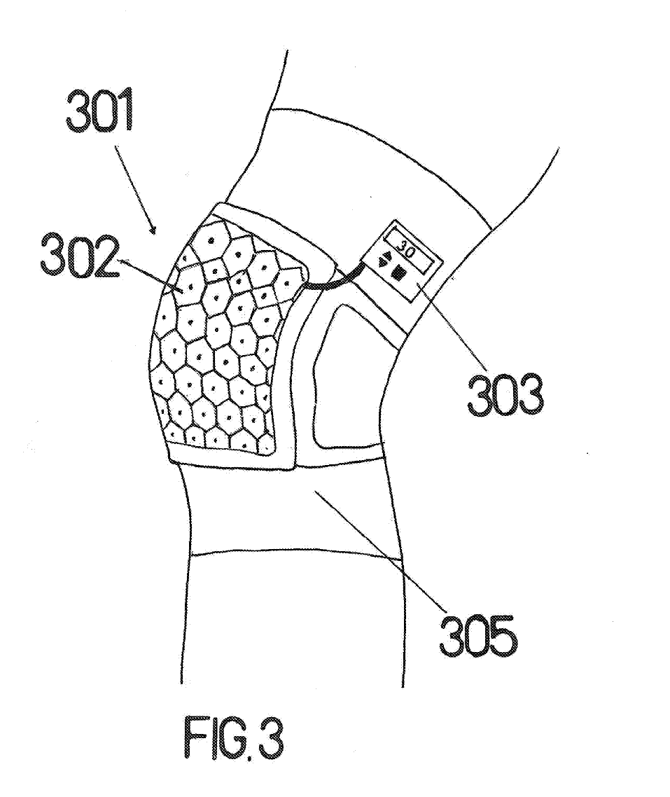

Another step that can be used to cause the fluid matrix to circulate adjacent articular cartridge is to use ultrasonic pressure waves to stimulate the fluid matrix and/or articular cartridge. Referring to FIG. 3, a human knee joint 301 is depicted with an ultrasonic transducer assembly 302 affixed to it, such as by use of hook and loop fastener means, adhesive, or an elastomeric band 305. The ultrasonic transducer assembly includes a power source such as a battery and a control unit 303, such as a computer or circuit board, for determining frequency, amplitude, and duration of ultrasonic waves emitted. The ultrasonic waves will stimulate the fluid matrix to contact chondrocytes in the articular cartilage, and will stimulate chondrocytes in the articular cartridge to uptake materials of the fluid matrix.

Frequency of ultrasonic waves produced by the ultrasonic transducer can be greater than 10,000 kHz, greater than 20,000 kHz, greater than 50,000 kHz, greater than 100,000 kHz, greater than 500,000 kHz, or greater than 1,000,000 kHz. Wavelength of the ultrasonic waves will typically be less than 1 cm, less than 1 millimeter, or less than 10 nanometer. Some wavelengths could include 5 nanometers, 2 nanometers and 1 nanometer. A wavelength can be chosen which provides a desired constituent of the matrix with greatest excitability or greatest mobility to encourage uptake by chondrocytes. Such excitability and mobility are believed to be maximized when a wavelength approximately equal to the diameter of a molecular structure to be excited or mobilized.

Ultrasonic treatment of the matrix to cause it to contact chondrocytes and be taken up by chondrocytes can include maintaining the ultrasonic waves for a predetermined period of time, such as 5 seconds, 10 seconds, 30 seconds, 1 minute, 3 minutes, 5 minutes, 10 minutes, etc., followed by a rest period. Alternatively, the ultrasonic transducer may be rapidly turned on and off to provided pulsed ultrasonic waves.

After the matrix and materials of the matrix have been moved toward chondrocytes such as by use of a pressure cuff or ultrasonic transducer, excitation of the chondrocytes themselves can be performed in order to increase their uptake of materials of the matrix. Excitation of the chondrocytes can be performed by exposing them to photons of a desired wavelength at an appropriate power setting. A range of possible wavelengths for such photonic exposure includes from about 165 nm to 280 nm. Example wavelengths in this range which may be particularly useful include 170 nm, 185 nm, 193 nm, 206 nm, 210 nm, 212 nm, 222 nm, 224 nm and 253 nm. Lasers, light emitting diodes, and lamps can be selected to provide the desired wavelength and amplitude of photon exposure. Photonic exposure excites the chondrocytes and improves their uptake of materials from the matrix. Delivery of the photons may be by continuous wave, pulsed or otherwise. Delivery may be for a period of seconds or minutes, followed by a rest, and then continued.

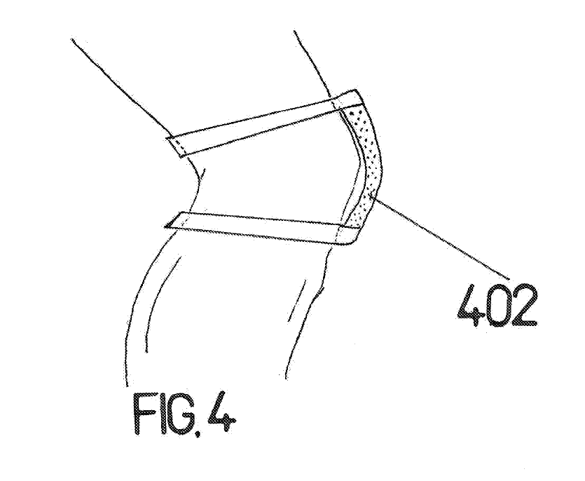

Referred to FIG. 4, a human knee 401 is depicted with a photonic exposure device 402 mounted to it. The photonic exposure device may be mobile so that it can be used throughout the day. The photonic exposure device includes a photon emitter such as an LED, lamp or laser, a power supply such as a battery, and a control unit such as a computer or circuit board. The photonic exposure device exposes chondrocytes of the articular cartridge to exciting photon energy of a desired wavelength and amplitude for a desired time period and according to a desired waveform. Exposure may be repeated. Exposure may be in conjunction with use of a pressure cuff or ultrasonic transducer or separately. The control unit can be programmed so that the patient's articular cartridge receives prescribed photonic exposure multiple times per day or night as needed to achieve maximum chondrocyte excitation.

The invention can be implemented in variety of different configurations as needed to tailor treatment to particular patient needs.

1. A method for encouraging in-situ articular cartilage re-growth through chondrocyte stimulation comprising the steps of:
(a) injecting a fluid matrix containing both (i) materials that chondrocytes can use for re-growth of articular cartilage, and (ii) materials that tend to encourage chondrocytes to re-grow articular cartilage,
(b) causing said fluid matrix to circulate adjacent chondrocytes so that both (i) said materials that chondrocytes can use for re-growth of articular cartilage and (ii) said materials that tend to encourage chondrocytes to re-grow articular cartilage come into contact with chondrocytes,
(c) expose said chondrocytes to photonic stimulation having a wavelength in the range of 165 nm to 280 nm in order to excite chondrocytes to uptake both (i) injected materials that chondrocytes can use for re-growth of articular cartilage and (ii) injected materials that tend to encourage chondrocytes to re-grow articular cartilage.

2. A method as recited in claim 1 where said step (b) is performed at least in part by use of a pressure cuff.

3. A method as recited in claim 1 where said step (b) is performed at least in part by use of an ultrasonic transducer.

4. A method as recited in claim 1 wherein at least one of said materials that chondrocytes can use for re-growth of articular cartilage is selected from the group consisting of lysine, proline, Vitamin C, copper peptides, and hormones.

5. A method as recited in claim 1 wherein at least one of said materials that tend to encourage chondrocytes to re-grow articular cartilage is selected from the group consisting of hyaluronic acid, nanosized carbon tubes, nanoparticulate Ti, nanoparticle CoCr, nanoparticle Ti6Al4V, nanoparticle hydroxyapatite, nanoparticle titanium, nanoparticle aluminum and nanoparticle zinc oxide.

6. A method as recited in claim 1 wherein at least one of said materials that tend to encourage chondrocytes to re-grow articular cartilage includes a naturally-occurring biological tissue growth regulator.

7. A method as recited in claim 1 wherein said photonic stimulation includes use of light having a wavelength selected from the group consisting of 170 nm, 185 nm, 193 nm, 206 nm, 210 nm, 212 nm, 222 nm, 224 nm and 253 nm.

8. A method as recited in claim 1 wherein said photonic stimulation is provided in a format of pulsed light.

9. A method as recited in claim 3 wherein an ultrasonic transducer is placed next to a joint that has been injected with said fluid matrix, and said ultrasonic transducer is powered to emit ultrasonic waves which tend to cause circulation of said injected fluid matrix.

10. A method as recited in claim 9 wherein exposure to said ultrasonic waves will stimulate chondrocytes in the articular cartridge to uptake materials of said injected fluid matrix.

11. A method as recited in claim 9 wherein the frequency of ultrasonic waves produced by said ultrasonic transducer is greater than 10,000 kHz.

12. A method as recited in claim 9 wherein the frequency of ultrasonic waves produced by said ultrasonic transducer is greater than 50,000 kHz.

13. A method as recited in claim 9 wherein the frequency of ultrasonic waves produced by said ultrasonic transducer is greater than 100,000 kHz.

14. A method as recited in claim 11 wherein said ultrasonic waves include a wavelength selected to approximately equal the diameter of at least one molecular structure included in said injected fluid matrix, and wherein said ultrasonic waves tend to drive said at least one molecular structure into chondrocytes.

15. A method as recited in claim 11 wherein said ultrasonic waves are produced in an intermittent on-off pattern for pre-determined time period.
